(19) United States
(12) Patent Application Publication
(10) Pub. No.: US 2020/0306025 A1
(43) Pub. Date: Oct. 1, 2020

(54) PACKAGE FOR STORING, PRE-PROCESSING, HANDLING, AND DELIVERING A MEDICAL DEVICE TO A SURGICAL SITE

(71) Applicant: SPINAL BALANCE, INC., Swanton, OH (US)

(72) Inventors: Faezah Agarwal, Swanton, OH (US); Aakash Agarwal, Swanton, OH (US)

(73) Assignee: SPINAL BALANCE, INC., Swanton, OH (US)

(21) Appl. No.: 16/765,894

(22) PCT Filed: Nov. 21, 2018
(86) PCT No.: PCT/US2018/062182

§ 371 Date: May 21, 2020

Related U.S. Application Data
(60) Provisional application No. 62/590,145, filed on Nov. 22, 2017.

(51) Int. Cl. A61F 2/00; A61B 50/30

(52) U.S. Cl. CPC A61F 2/0095; A61B 2050/0058; A61B 2050/3005; A61B 50/30

(57) ABSTRACT

A package for a medical device includes a first portion and a second portion. The first portion is adapted to support a medical device having a receiving area. The second portion has an area that is adapted to be aligned with the receiving area of the medical device supported in the first portion. The area of the second portion can be deformed to move a medical material into the receiving area of the medical device. A method of providing a medical device with a medical material includes the steps of: (a) providing a package including a first portion containing a medical device; (b) providing a medical material; and (c) deforming a second portion of the package to move the medical material into or onto the medical device.

### CROSS-REFERENCE TO RELATED APPLICATIONS

This application claims the benefit of U.S. Provisional Application No. 62/590,145, filed Nov. 22, 2017, the disclosure of which is incorporated herein by reference.

### BACKGROUND OF THE INVENTION

This invention relates in general to packaging structures for medical devices. In particular, this invention relates to an improved package for storing, pre-processing, handling, and delivering a medical device to a surgical site for use.

Many medical devices require cumbersome pre-processing at an operation site before they are ready for implantation. In most instances, the handling and assistive delivery of such implants is crucial for both the surgeon or other medical user and the patient. For the surgeon or other user, it is desirable to provide savings in surgical time, better ergonomics, and ease and assistance during surgeries. For patients, it desirable to allow for less blood loss, superior asepsis, less chance of post-surgery complications, and expedited healing.

These medical devices are typically delivered to the operation site either in a steam pre-processed tray or in a pre-sterilized package, such as a plastic container, tray, peel pouch, etc. The medical devices may include, for example, interbody cages or breast implants and may be pre-loaded on an instrument used for quick delivery at the surgical site. The pre-processing may involve hydrating, mixing, or packing of some kind of substance onto or into the medical device, which allows it to function in a desired manner, or at least increases its efficacy.

At present, the packing of a bone graft or other medical material onto or into the medical device, or hydrating the medical device, requires a user to perform multiple manual steps, which may contaminate the medical device and/or the medical material. These manual steps primarily include (1) the initial filling of the medical material onto or into the medical device, and (2) the subsequent compacting (or exposure and homogenization, in the case of hydration) of the medical material onto or into the medical device. The first step is usually accomplished manually by the surgeon or other user using his or her fingers using an instrument such as a scapula (for cases involving a solid material) or a syringe (for cases involving a fluid material). The second step is usually accomplished manually by the surgeon or other user using his or her fingers or a tamping instrument. Initial delivery/exposure using fingers and compacting/homogenization through finger pressure is the currently preferred method and is currently the most practical because of the ease in maneuverability and the ability to fill/expose, confine/retain, and compact/homogenize the medical material at the same time.

The disadvantage of this process, however, is that multiple touches by the surgeon or other user to both the medical device and the medical material are required. The gloves of the surgeon or other user are known to harbor bacteria (such as from the wound site), and reusable surgical instruments (such as tamps) have been shown to host foreign body compositions, such as endotoxins, carbohydrates, etc. Furthermore, a tabletop surface, which is often used to hold the implant and/or the medical material prior to use, may contain blood spills, tissues (such as retrieved from the patient during surgery), etc. It is also known that if a surgeon or other user elects to reduce the number of those touches by using instruments, then he or she is left with a reduced ability to maneuver (e.g., fill/exposure and compact/homogenize), which can increase surgical time, may still contaminate the device, and/or perform an inadequate pre-processing, leading to failure of function or efficacy of the medical device. Therefore, the common practice remains the use of fingers for pre-processing and handling the medical device.

The missing link in the art of packaging, handling, and delivery of such medical devices is a system that provides for both reduction or elimination of direct contact by the surgeon or other user and ease in filling and compacting of the medical device. An ideal packaging would allow ease of pre-processing of the medical device, while minimizing the handling, contamination, and time loss before delivery to the patient.

Thus, it would be desirable to provide an improved package for storing, pre-processing, handling, and delivering of a medical device to a surgical site for use that addresses these issues.

### SUMMARY OF THE INVENTION

This invention relates to an improved package for storing, pre-processing, handling, and delivering of medical device to a surgical site for use.

Disclosed herein is a package and method for containment of a medical device that also helps in the initial filling of desired material and its compaction, homogenization, or dispersion into the medical device using ergonomic maneuvers and without the surgeon or other user directly touching the medical device or the medical material as it is being filled in during the process. The invention allows surgeons and other users to effortlessly (i.e., without using a tamp or other instrument) fill and/or compact and/or disperse and/or homogenize the medical material onto or into the medical device without having to touch it.

In its most basic embodiment, the package contains a cavity to contain the medical device and multiple access points to hold the package (and the medical device) and attach an instrument to the medical device for filling the medical material onto or into the medical device and for retrieving the medical device, and pliable or combination of pliable and non-pliable features to fill and/or compact and/or disperse and/or homogenize the added substance onto or into the medical device.

Various aspects of this invention will become apparent to those skilled in the art from the following detailed description of the preferred embodiments, when read in light of the accompanying drawings.

### BRIEF DESCRIPTION OF THE DRAWINGS

FIG. 1 is a perspective view of a first embodiment of an improved medical device package in accordance with this invention including a lid shown in a closed position relative to a base.

FIG. 2 is a perspective view of the first embodiment of the medical device package illustrated in FIG. 1 shown with a sterilized medical device enclosed therein.

FIG. 3 is a top plan view of the first embodiment of the medical device package illustrated in FIG. 2 showing the lid in an opened position relative to the base.

FIG. 4 is a side elevational view of the first embodiment of the medical device package illustrated in FIG. 3 showing a medical material deposited in a retaining area provided on the lid.

FIG. 5 is a side elevational view of the first embodiment of the medical device package illustrated in FIG. 4 showing the base in the closed position relative to the lid.

FIG. 6 is a side elevational view of the first embodiment of the medical device package illustrated in FIG. 5 showing the retaining area of the lid after being deformed.

FIG. 7 is a side elevational view of the first embodiment of the medical device package illustrated in FIG. 6 showing the lid in the opened position relative to the base.

FIG. 8 is a top plan view of the first embodiment of the medical device package illustrated in FIG. 7.

FIG. 9 is a top plan view of the first embodiment of the medical device package illustrated in FIG. 8 after a handling tool has been connected to the medical device in the medical device package.

FIG. 10 is a perspective view of a second embodiment of an improved medical device package in accordance with this invention showing an inner housing in an extended position relative to an outer housing and having a medical device disposed therein.

FIG. 11 is a perspective view of the second embodiment of the medical device package illustrated in FIG. 10 showing a medical material deposited in the medical device.

FIG. 12 is a side elevational view of the second embodiment of the medical device package illustrated in FIGS. 10 and 11 showing the inner housing in a retracted position relative to the outer housing.

FIG. 13 is a perspective view of the second embodiment of the medical device package illustrated in FIG. 12 showing the inner housing in the retracted position relative to the outer housing and showing an area of the base after being deformed.

### DETAILED DESCRIPTION OF THE PREFERRED EMBODIMENTS

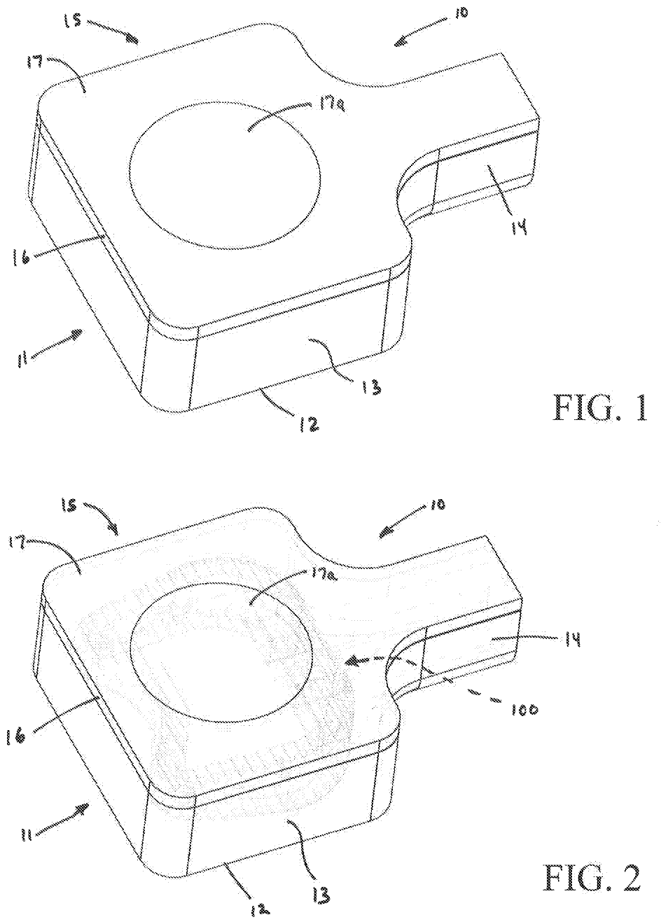

Referring now to the drawings, there is illustrated in FIGS. 1 through 9 a first embodiment of an improved medical device package, indicated generally at 10, in accordance with this invention. As shown therein, the package 10 is intended to function as an enclosure for a medical device, indicated generally at 100. The illustrated medical device 100 is a conventional implant that is adapted to be inserted between adjacent vertebrae (not shown) in a spinal column for the purpose of fusing same together. To that end, the medical device 100 has a receiving area 100a provided therein for a purpose that will be explained in detail below. Alternatively, the medical device 100 may be a hydrating medical device, a vessel occlusive device, a tissue expander, or a breast implant, for example. However, the illustrated medical device 100 is intended merely to illustrate one application for which this invention may be used. Thus, the package 10 of this invention is not intended to be limited for use with the specific structure for the illustrated medical device 100 or with medical devices in general. On the contrary, as will become apparent below, this invention may be used in any desired environment and for any desired purpose.

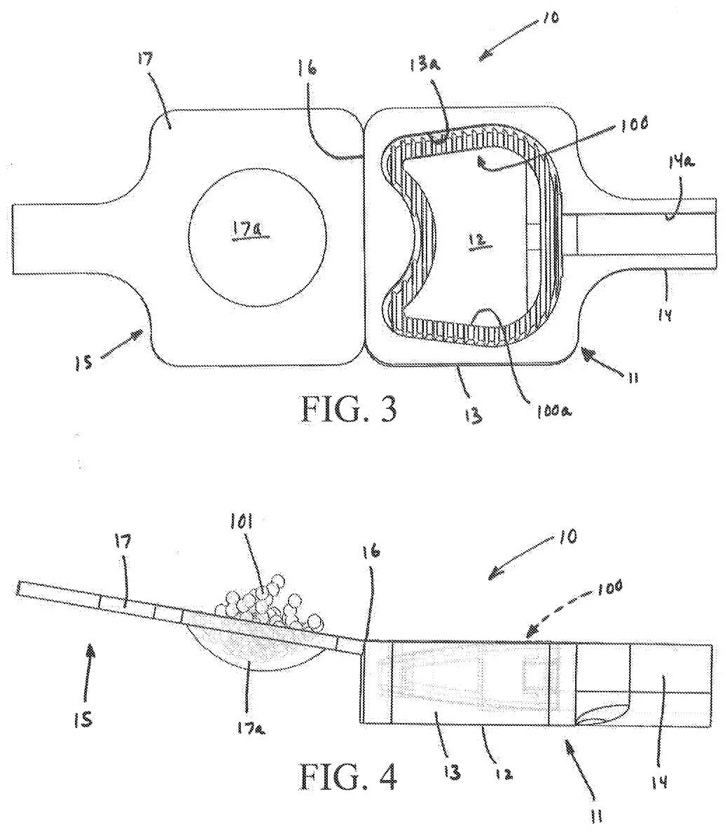

The structure of the first embodiment of the recessed area package 10 is best illustrated in FIGS. 1 through 4. As shown therein, the package 10 includes a base, indicated generally at 11, that functions as a first portion of the enclosure for the medical device 100. The illustrated base 11 is generally rectangular shape and includes a bottom wall 12 and a side wall 13 that together define the first portion of the enclosure for the medical device 100. As best shown in FIG. 3, the illustrated side wall 13 has an inner surface 13a that is shaped in conformance with an outer surface of the medical device 100. Such cooperating shapes function to positively position the medical device 100 relative to the base 11 of the package 10, as clearly shown in FIG. 3. However, the bottom wall 12 and the inner surface 13a of the side wall 13 of the base 11 may have any desired shapes, and the shape of the inner surface 13a of the side wall 13 need not conform to the shape of the outer surface of the medical device 100. The package 10 also includes an extension portion 14 extending from the side wall 13 of the base 11. The extension portion 14 of the package 10 has a recessed area 14a that is provided therein for purpose that will be explained below.

The package 10 further includes a lid 15 that functions as a second portion of the enclosure for the medical device 100. In the illustrated embodiment, the lid 15 is formed integrally with the base 11 of the package 10 so as to define a living hinge 16 therebetween. However, the lid 15 of the package 10 may be connected to or otherwise supported on the base 11 of the package 10 in any desired manner. Alternatively, the lid 15 of the package 10 may be formed separately from the base 11 of the package 10. The illustrated lid 15 of the package 10 includes an upper wall 17 having a recessed area 17a provided therein. As best shown in FIG. 4, the recessed area 17a of the upper wall 17 is circular in shape and is concave relative to the interior of the package 10, although neither is required. Preferably, the recessed area 17a is formed integrally with the other portions of the upper wall 17 and is relatively flexible compared thereto. However, the recessed area 17a may be formed separately from the upper wall 17 of the lid 15 if desired.

FIGS. 1 and 2 show the lid 15 in a closed position relative to the base 11, wherein the base 11 and the lid 15 cooperate to form the enclosure for the medical device 100. However, as shown in FIGS. 3 and 4, the lid 15 may be moved to an opened relative to the base 11, wherein the base 11 and the lid 15 are spaced apart from one another to provide access to the medical device 100 disposed within the enclosure.

The operation and manner of use of the first embodiment of the package 10 will now be described with reference to FIGS. 1 through 9. Initially, both the package 10 and the medical device 100 are sterilized in any conventional manner. Then, the medical device 100 is disposed within the base 11 of the package 10, and the lid 15 is moved to the closed position shown in FIG. 2. In this manner, the medical device 100 can be transported within the package 10 in its sterilized condition to a desired location, such as a surgical operating site.

Next, when it is desired to use the medical device 100, the lid 15 of the package 10 is moved to the opened position relative to the base 11, as shown in FIG. 3. Typically, this will occur only immediately before the medical device 100 is needed for use. Regardless, after the lid 15 is moved to the opened position and before the medical device 100 is removed from the base 11 of the package 10, a quantity of a medical material 101 is disposed in the recessed area 17a provided in the upper wall 17 of the lid 15, as shown in FIG. 4. The medical material 101 is, of itself, conventional in the art and forms no part of this invention. Non-limiting examples of the medical material 101 include natural bone graft and synthetic osteoconductive, osteoinductive, or osteogenetic materials. Such medical material may also be a hydrating liquid available in various delivery forms (such as by syringe, vial, or bowl), bone grafts, or any substitutes, which may be either retrieved or morselized on site or are available in pre-packaged forms, etc. The medical material 101 is preferably disposed in the recessed area 17a using a sterile tool (not shown), as opposed to being touched by the gloved fingers of the surgeon or other user.

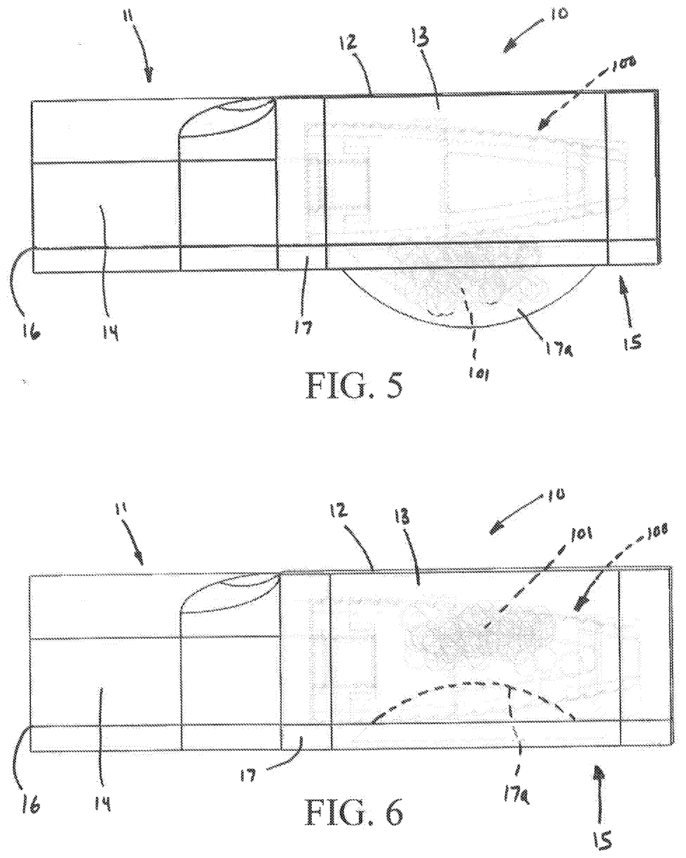

After the medical material 101 has been disposed in the recessed area 17a, the base 11 of the package 10 is then moved from the opened position relative to the lid 15 to be closed position, as shown in FIG. 5. Preferably, this is accomplished by maintaining the lid 15 stationary and pivoting the base 11 relative thereto. Such movement, which is not required, utilizes the force of gravity to maintain the medical material 101 in the recessed area 17a of the lid 17 and, thus, minimizes the opportunity for the medical material 101 to spill or otherwise undesirably move out of the recessed area 17a. As shown in FIG. 5, the package 10 is inverted from its original orientation shown in FIGS. 1 and 2, although such is not required. In this condition, the medical material 101 in the recessed area 17a is preferably aligned with the receiving area 100a of the medical device 100.

Next, as shown in FIG. 6, the recessed area 17a of the lid 17 is deformed inwardly toward the medical device 100 disposed within the package 10 (i.e., to be convex relative to the interior of the package 10). Such deformation is preferably accomplished by the fingers of the surgeon or other user pressing (upwardly when viewing FIGS. 5 and 6) on the outer (bottom) surface of the lid 17. Because the medical material 101 in the recessed area 17a is aligned with the receiving area 100a of the medical device 100, such deformation of the lid 17 causes the medical material 101 to be moved out of the recessed area 17a and into the aligned receiving area 100a of the medical device 100. Importantly, this transfer of the medical material 101 from the recessed area 17a of the lid 17 into the receiving area 100a of the medical device 100 occurs without the medical material 101 being touched by the gloved fingers of the surgeon or other user.

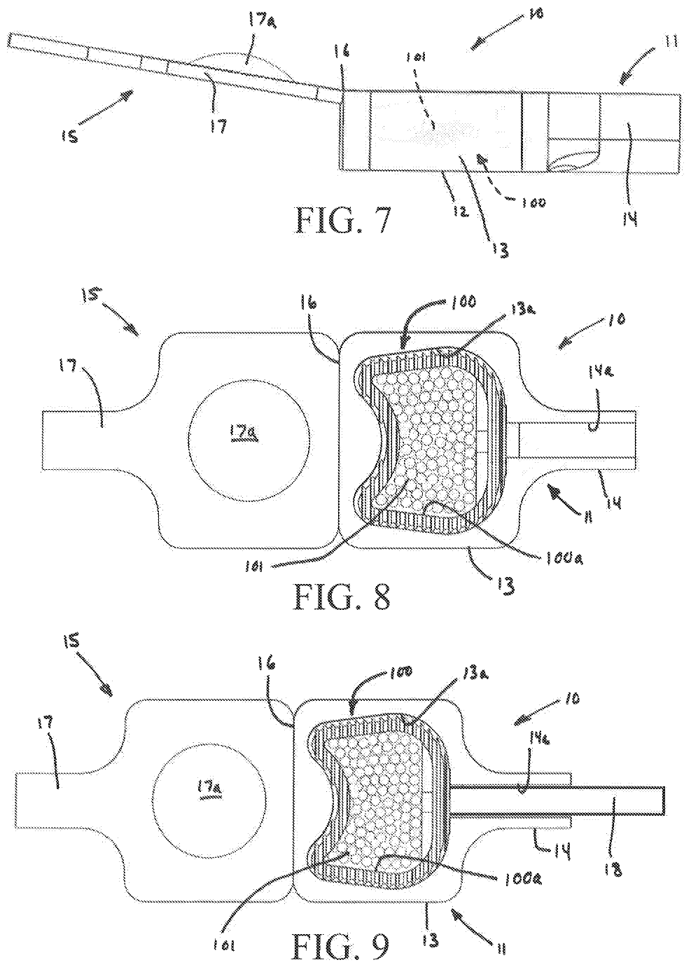

After the medical material 101 has been moved into the receiving area 100a of the medical device 100, the lid 17 is again moved from the closed position relative to the base 11 to the opened position, as shown in FIGS. 7 and 8. FIG. 8 best illustrates how the medical material 101 has been transferred into the receiving area 100a of the medical device 100. Lastly, as shown in FIG. 9, a tool 18 (which is manually grasped and manipulated by the surgeon or other user) is inserted through the recessed area 14a of the extension portion 14 of the base 11 into releasable engagement with a portion of the medical device 100. The tool 17 can be used to withdraw the medical device 100 (together with the medical material 101 contained therein) from the base 11 of the package 10. The tool 17 can further be used to transport the medical device 100 containing the medical material 101 from the package 10 to the surgical site for use. Once the medical device 100 is properly positioned at the surgical site, the tool 17 can be disengaged from medical device 100 and discarded.

Thus, it can be seen that the package 10 of this invention allows a medical material 101 to be inserted within a receiving area 100a of a medical device 100, and further allows the medical device 100 to be removed from the package 10 and implanted at a surgical site for use, all without the medical device 100 or the medical material 100a having been touched by the gloved fingers of a surgeon or other user. This significantly reduces the opportunity for contamination of either the medical device 100 or the medical material 100a to occur.

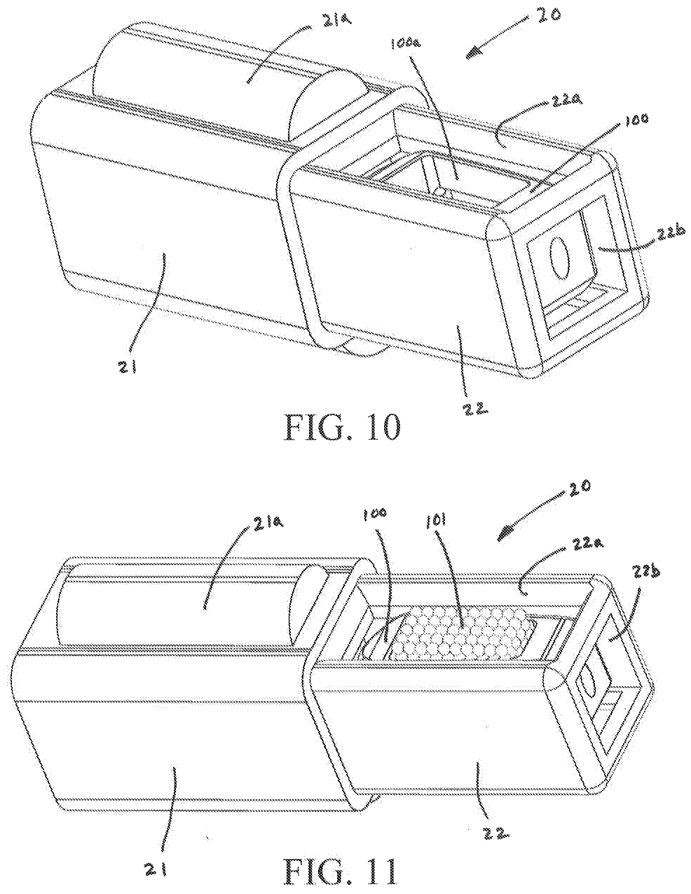

FIGS. 10 through 13 illustrate a second embodiment of an improved medical device package, indicated generally at 20, in accordance with this invention. The second embodiment of the package 20 includes an outer housing 21 that, in the illustrated embodiment, is shaped generally in the form of a hollow rectangular parallelepiped. However, the outer housing 21 may be formed having any desired shape. The outer housing 21 has a recessed area 21a provided therein. As best shown in FIGS. 10, 11, and 12, the recessed area 21a is initially concave relative to the interior of the outer housing 21, although such is not required.

The second embodiment of the package 20 also includes an inner housing 22 that is movable relative to the outer housing 21. In the illustrated embodiment, the inner housing 22 is also shaped generally in the form of a rectangular parallelepiped, although again such is not required. Preferably, however, the inner housing 22 has an outer surface that generally corresponds in shape with an inner surface of the hollow outer housing 21. As such, the outer surface of the inner housing 22 is supported on the inner surface of the outer housing 21 for lateral sliding movement relative thereto. However, the inner housing 22 may be supported on the outer housing 21 in any desired manner. The inner housing 22 has an opening 22a through an outer surface that extends into an interior space 22b. The purposes of the opening 22a and the interior space 22b will be explained below.

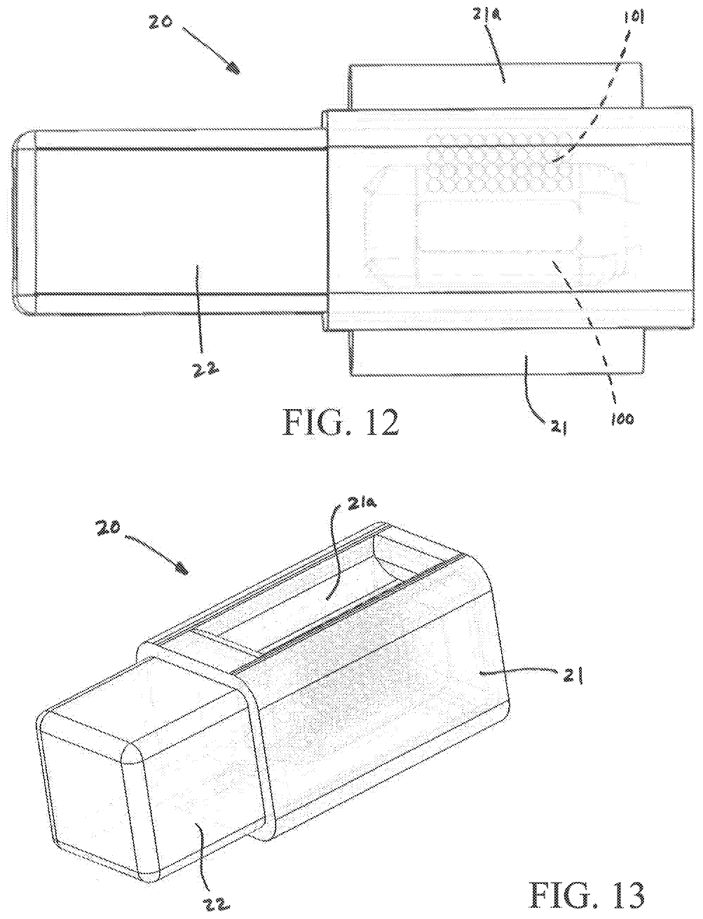

FIGS. 10 and 11 show the inner housing 22 in an extended position relative to the outer housing 21. In this extended position, the interior space 22b of the inner housing 22 is extended from the interior of the outer housing 21. FIGS. 12 and 13 show the inner housing 22 in a retracted position relative to the outer housing 21. In this retracted position, the interior space 22b of the inner housing 22 is disposed within the interior of the outer housing 21. For a reason that will become apparent below, when the inner housing 22 is in the retracted position relative to the outer housing 21, the opening 22a of the inner housing 22 is aligned with the recessed area 21a provided on the outer housing 21.

The operation and manner of use of the second embodiment of the package 20 will now be described with reference to FIGS. 10 through 13. Initially, both the package 20 and the medical device 100 are sterilized in any conventional manner. Then, the medical device 100 is disposed within the interior space 22b of the inner housing 22, as shown in FIG. 10, and the assembly of the package 20 and the medical device 100 is enclosed within a protective packaging device (not shown). In this manner, the package 20 and the medical device 100 can be transported in their sterilized conditions to a desired location, such as a surgical operating site.

When it is desired to use the medical device 100, the protective packaging device is removed, and the inner housing 21 is moved to the extended position shown in FIG. 10 (if it is not already provided in that orientation). Next, a quantity of the medical material 101 is disposed within the receiving area 100a of the medical device 100, as shown in FIG. 11. The medical material 101 is preferably disposed in the receiving area 100a of the medical device 100 using a sterile tool (not shown), as opposed to being touched by the gloved fingers of the surgeon or other user. Then, the inner housing 22 is moved from the extended position shown in FIG. 11 to the retracted position shown in FIG. 12. When this occurs, when medical material 100 is preferably aligned with the recessed area 21a of the outer housing 21, as also shown in FIG. 12.

Next, as shown in FIG. 13, the recessed area 21a of the outer housing 21 is deformed inwardly toward medical device 100 disposed within the package 20 (i.e., to be convex relative to the interior of the package 20). Such deformation is preferably accomplished by the fingers of the surgeon or other user pressing (downwardly when viewing FIGS. 12 and 13) on the outer (upper) surface of the recessed area 21a of the outer housing 21. Because the medical material 100 in the recessed area 21a is aligned with receiving area 100a of the medical device 100, such deformation of the recessed area 21a causes the medical material 101 to be moved out of the recessed area 21a and into the aligned receiving area 100a of the medical device 100. Importantly, this transfer of medical material 101 from the recessed area 21a of the outer housing 21 into the aligned receiving area 100a of the medical device 100 occurs without the medical material 101 being touched by the gloved fingers of the surgeon or other user.

After the medical material 101 has been moved into the receiving area 100a of the medical device 100, the inner housing 22 is moved from the retracted position relative to the outer housing 21 to be extended position shown in FIGS. 10 and 11. Lastly, a tool (not shown) may be used to withdraw the medical device 100 (together with the medical material 101 contained therein) from the outer housing 21 and transport it to the surgical site for use. Thus, it can be seen that the package 20 of this invention also allows a medical material 101 to be inserted within a receiving area 100a of a medical device 100, and further allows the medical device 100 to be removed from the package 10 and implanted at a surgical site for use, all without either the medical device 100 or the medical material 100a being touched by the gloved fingers of a surgeon or other user.

The principle and mode of operation of this invention have been explained and illustrated in its preferred embodiments. However, it must be understood that this invention may be practiced otherwise than as specifically explained and illustrated without departing from its spirit or scope.

What is claimed is:

1. A package for a medical device comprising:
a first portion that is adapted to support a medical device having a receiving area; and
a second portion having an area that is adapted to be aligned with the receiving area of the medical device supported in the first portion,
wherein the area of the second portion can be deformed to move a medical material into the receiving area of the medical device.

2. The package defined in claim 1 wherein the area of the second portion is a recessed area that is adapted to receive a medical material, and wherein the recessed area of the second portion can be deformed to move the medical material from the recessed area of the second portion into the receiving area of the medical device.

3. The package defined in claim 1 wherein the first portion is a base, and the second portion is a lid that is movable relative to the base.

4. The package defined in claim 3 wherein the lid is connected to the base by a living hinge.

5. The package defined in claim 3 wherein the area of the second portion is a recessed area formed on the lid that is concave relative to an interior of the package.

6. The package defined in claim 1 wherein the first portion is an inner housing, and the second portion is an outer housing that is movable relative to the inner housing.

7. The package defined in claim 6 wherein the inner housing is slidably supported on the outer housing for movement between an extended position and a retracted position.

8. The package defined in claim 6 wherein the area of the second portion is a recessed area formed in the outer housing that is concave relative to an interior of the package.

9. A method of providing a medical device with a medical material comprising the steps of:
(a) providing a package including a first portion containing a medical device;
(b) providing a medical material; and
(c) deforming a second portion of the package to move the medical material into or onto the medical device.

10. The method defined in claim 9 wherein step (a) is performed by providing the package with a base that contains the medical device, and wherein step (b) is performed by providing the medical material on a lid that is attached to the base.

11. The method defined in claim 10 wherein step (b) is performed by providing the medical material in a recessed area provided on the lid, and wherein step (c) is performed by deforming the recessed area.

12. The method defined in claim 9 wherein step (a) is performed by providing the package with an inner housing that contains the medical device, and wherein step (b) is performed by providing the medical material on an outer housing that supports the inner housing for sliding movement relative thereto.

13. The method defined in claim 12 wherein step (b) is performed by providing the medical material in a recessed area provided the outer housing, and wherein step (c) is performed by deforming the recessed area.
